(19) United States
(12) Patent Application Publication
(10) Pub. No.: US 2023/0079003 A1
(43) Pub. Date: Mar. 16, 2023

(54) MICROEMULSION BASE FOR IMPROVED PIGMENT ABSORPTION AND RELATED METHODS

(71) Applicant: KEMIN INDUSTRIES, INC., Des Moines, IA (US)

(72) Inventors: Krishna Gopal Dongol, Singapore (SG); Leow Sai Kaw, Skudai (MY); Ying Sin Koo, Singapore (SG); Tey Peck Ling, Singapore (SG)

(73) Assignee: KEMIN INDUSTRIES, INC., Des Moines, IA (US)

(21) Appl. No.: 17/856,145

(22) Filed: Jul. 1, 2022

Related U.S. Application Data
(60) Provisional application No. 63/217,382, filed on Jul. 1, 2021.

(51) Int. Cl. A23K 20/179; A23K 40/00; A23K 50/80; A23K 50/75

(52) U.S. Cl. CPC A23K 20/179; A23K 40/00; A23K 50/80; A23K 50/75

(57) ABSTRACT

The present invention relates to a microemulsion base that is capable of functioning as a self-emulsifying microemulsion base and does not require the addition of water or an aqueous phase. The compositions of the present invention can be directly applied to saponified pigments to form a stable microemulsion with improved pigment absorption. Another aspect of the present invention relates to the process for creating stable microemulsions with improved pigment absorption. Another aspect of the present invention relates to methods of using stable microemulsions to deliver pigments or carotenoids to animals to impart preferred attributes to animals. Another aspect of the present invention relates to a two-in-one microemulsion system with enhanced delivery, solubility and absorption of a pigment or carotenoid.

### CROSS-REFERENCE TO RELATED APPLICATIONS

The present application claims the benefit of priority to U.S. Provisional Patent Application No. 63/217,382, filed Jul. 1, 2021, entitled “MICROEMULSION BASE FOR IMPROVED PIGMENT ABSORPTION AND RELATED METHODS,” the entire disclosure of which is hereby incorporated by reference in its entirety.

### BACKGROUND OF THE INVENTION

Pigments are widely used in the food industry to enhance pigmentation of the skin of broilers and egg yolk color. However, many pigments, such as carotenoids and xanthophylls, are water insoluble, limiting the solubility and absorption in the animals. As such, in order to achieve the desired outcome, a higher dose of pigments is necessary, which increases the overall cost. Accordingly, the price of natural or plant-based pigments, such as carotenoids, and specifically lutein, is often too expensive for certain applications, and thus, not cost effective at increased dosage ranges.

Quantum GLO™ (Kemin Industries) is a commercially available product that contains a microemulsion to improve solubility and absorption of xanthophylls. The microemulsion system contains a synthetic surfactant, water and oil phase with maximum concentration of actives at 20 g/kg. Despite the benefits of this product, there remains a need for a highly concentrated microemulsion that can be included in a smaller quantity yet provide improved bioavailability and absorption, for instance, a new microemulsion able to encapsulate higher >40 g/kg xanthophyll.

In order to overcome these existing limitations, the inventors have identified a novel, highly-concentrated microemulsion base that improves the solubility of water insoluble pigments. This improvement allows for inclusion of pigments that have traditionally been limited in applications, where greater amounts were previously required. More specifically, the inventors have surprisingly found that the novel microemulsion base can be applied to saponified pigments and the water within the saponified pigments form a stable microemulsion.

### SUMMARY OF THE INVENTION

The present invention relates to compositions that contain a microemulsion base that does not require an aqueous phase. The inventors have surprisingly identified that the compositions of the present invention can be combined with saponified pigments to form a stable microemulsion with improved pigment absorption. Another aspect of the present invention relates to compositions with a high concentration of bio-emulsifier/surfactants that are capable of functioning as a “self-emulsifying” microemulsion base. Another aspect of the present invention relates to a method of delivering at least one carotenoid, pigment and/or other water-insoluble bioactive ingredient or nutrient to an animal by combining a microemulsion base that contains an oil phase, one or more non-ionic surfactants, one or more co-surfactants, and a bio emulsifier, with at least one carotenoid, pigment and/or other water-insoluble bioactive ingredient or nutrient.

Another aspect of the present invention relates to the process for creating stable microemulsions with improved pigment absorption. Another aspect of the present invention relates to methods of using stable microemulsions to impart preferred attributes to animals or fish, such as improved pigment absorption. The microemulsion base of the present invention addresses the issue of low solubility and bioavailability of water insoluble pigments in-vivo. Yet another aspect of the present invention relates to a two-in-one microemulsion system with enhanced delivery, solubility and absorption of pigment.

### DETAILED DESCRIPTION OF THE FIGURES

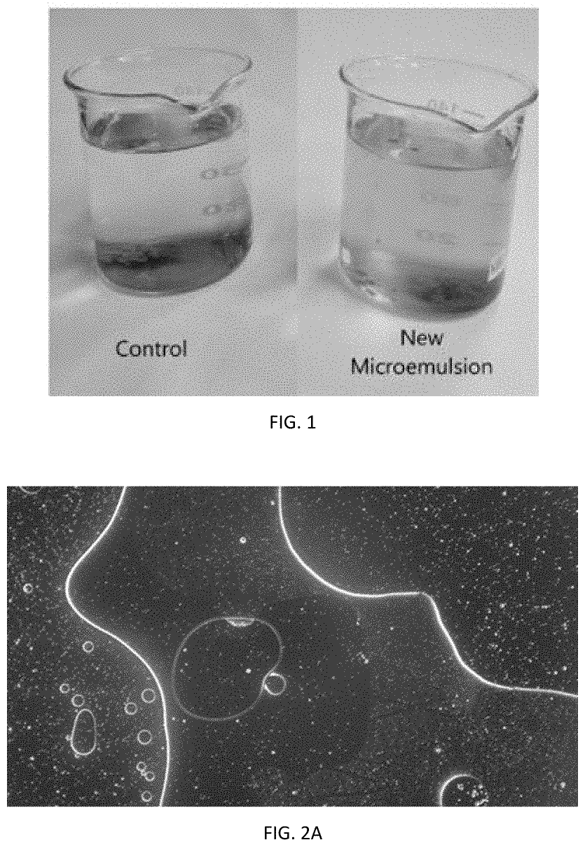

FIG. 1 depicts the dispersion of pigment in water after 1 hour, where the sample containing the microemulsion shows dispersion of the pigment in water.

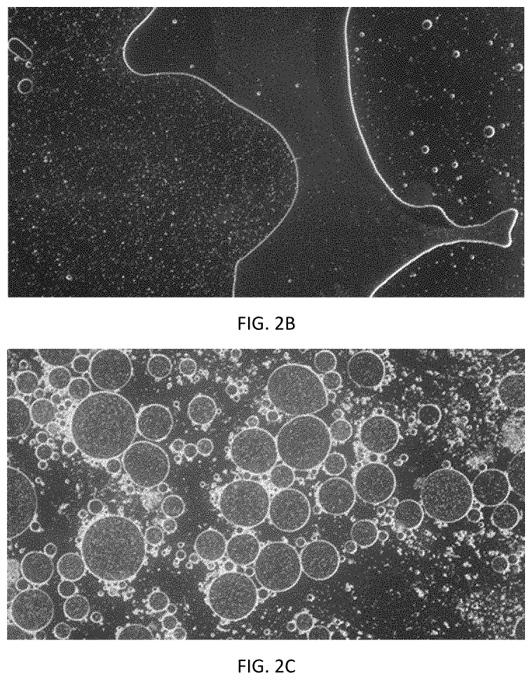

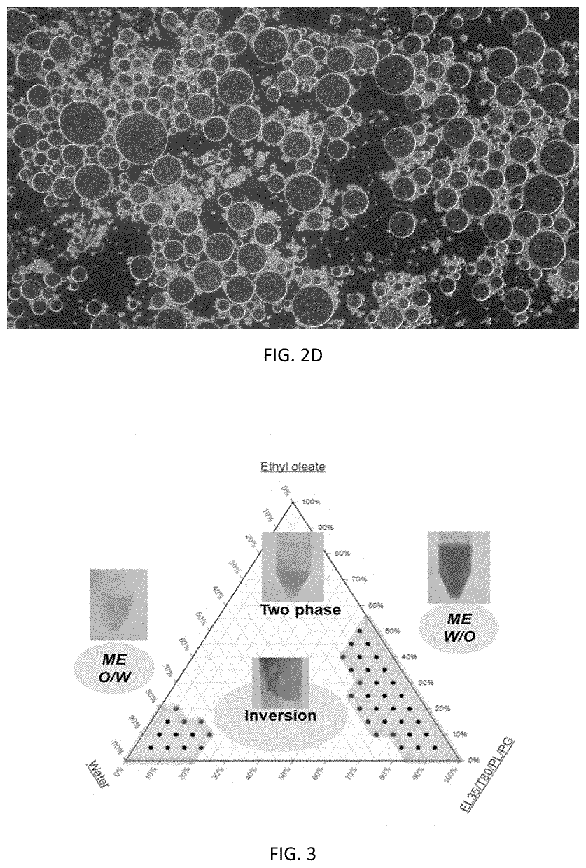

FIG. 2A depicts a microscopic image of a water and oil mixture with no microemulsion base FIG. 2B depicts a microscopic image of a water and oil mixture with no microemulsion base

FIG. 2C depicts a microscopic image of water and oil mixture with added microemulsion base.

FIG. 2D depicts a microscopic image of water and oil mixture with added microemulsion base.

FIG. 3 is a phase diagram, ME w/o area represents the desired combination that result in clear microemulsion (ME) and ME o/w area represent an area of emulsion

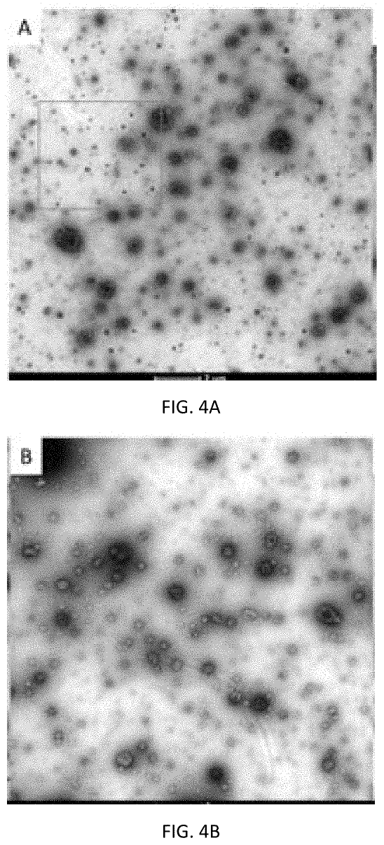

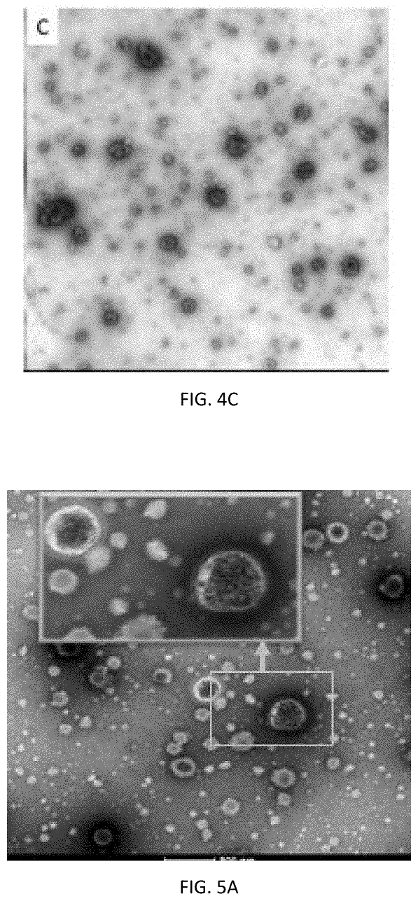

FIG. 4A depicts a representative TEM micrograph (2 μm) microscopic image of saponified concentrate (“sap con”) with new microemulsion base.

FIG. 4B depicts a representative TEM micrograph (2 μm) microscopic image sap conc only

FIG. 4C depicts a representative TEM micrograph (2 μm) microscopic image of sap conc with existing microemulsion.

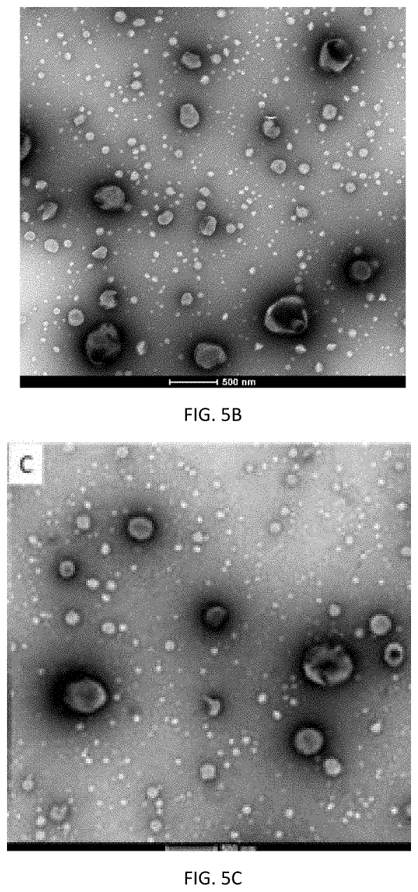

FIG. 5A depicts representative TEM micrographs (500 nm) microscopic image of sap conc with new microemulsion base.

FIG. 5B depicts representative TEM micrographs (500 nm) microscopic image sap conc only.

FIG. 5C depicts representative TEM micrographs (500 nm) microscopic image of sap conc with current microemulsion.

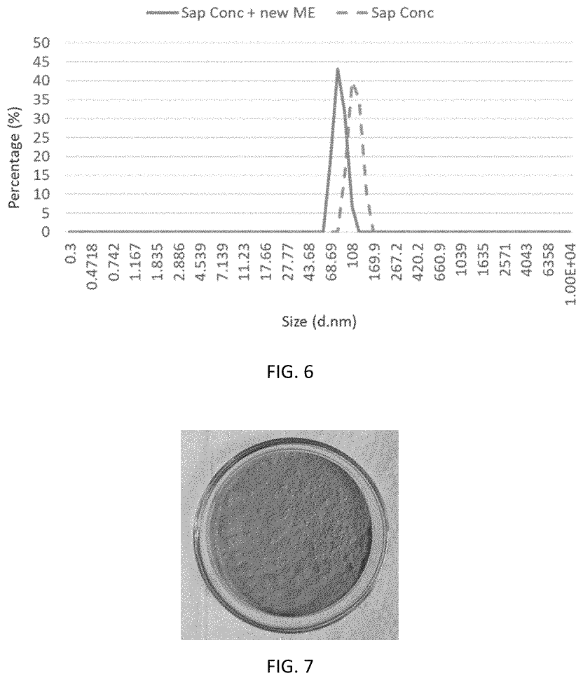

FIG. 6 depicts the size distribution of sap conc with new microemulsion and sap conc only.

FIG. 7 is a photo of the Quantum Glo 40 prototype, which is a gold-orange color.

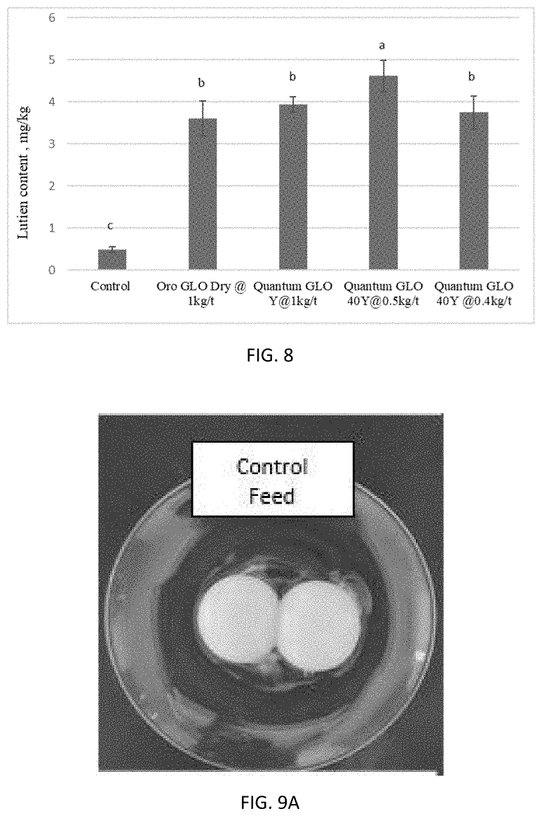

FIG. 8 depicts the lutein content comparison of each treatment groups.

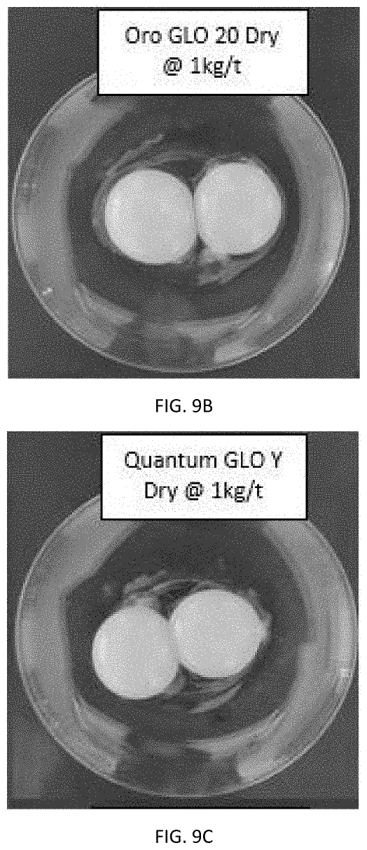

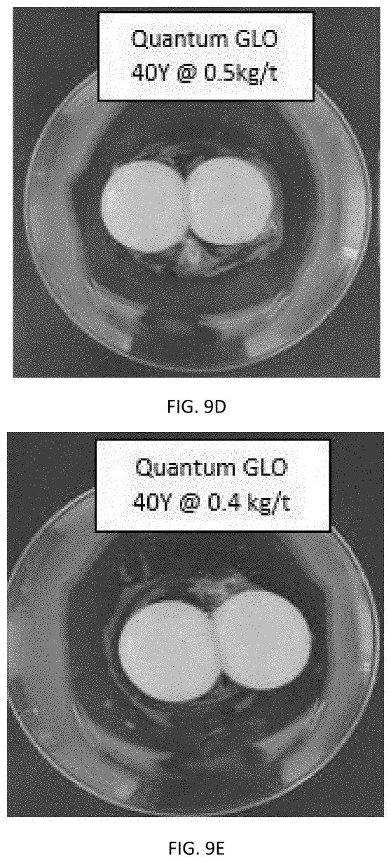

FIG. 9A is a photo depicting the varying Yolk Color of different treatments, with and without (Control) pigment in feed.

FIG. 9B is a photo depicting the varying Yolk Color of different treatments, with and without (Control) pigment in feed.

FIG. 9C is a photo depicting the varying Yolk Color of different treatments, with and without (Control) pigment in feed.

FIG. 9D is a photo depicting the varying Yolk Color of different treatments, with and without (Control) pigment in feed.

FIG. 9E is a photo depicting the varying Yolk Color of different treatments, with and without (Control) pigment in feed.

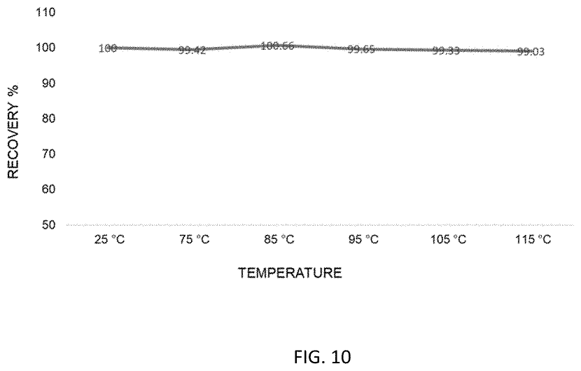

FIG. 10 depicts the recovery of Quantum GLO 40 at various temperatures.

### DETAILED DESCRIPTION OF THE INVENTION

The present invention relates to compositions with high concentration of bio-emulsifier/surfactants that are capable of functioning as a self-emulsifying microemulsion base. The compositions of the present invention can be directly applied to saponified pigments to form a stable microemulsion with improved pigment absorption. Another aspect of the present invention relates to the process for creating stable microemulsions with improved pigment absorption. Another aspect of the present invention relates to methods of using stable microemulsions to impart preferred attributes, such as improved pigment absorption. Another aspect of the present invention relates to a two-in-one microemulsion system with enhanced delivery, solubility and absorption of pigment.

According to at least one embodiment, the present invention is a self-emulsifying microemulsion base containing a system of using a long chain fatty acid ester as oil phase, such as ethyl oleate, one or more food grade ethoxylated non-ionic surfactants (such as EL 35, generally understood as polyoxyl 35 castor oil or ethoxylated castor oil, and Tween 80, generally understood as polysorbate 80 or polyethylene sorbitol ester), hydrolyzed and unhydrolyzed lecithin, and at least one short chain alcohol co-surfactant, such as propylene glycol. In certain embodiments, the recited ingredients are present in an amount disclosed in Table 1. By way of non-limiting example, for instance, at roughly 5-8% inclusion of the microemulsion base with saponified pigment, the composition can form a stable microemulsion with about 7-8% water present within the saponified pigment and emulsify over 50% saponified pigment.

TABLE 1
The composition of the microemulsion base prototype.

| Ingredient | Composition (%) | Function |
|---|---|---|
| Ethyl oleate | 15.00 | Oil base |
| EL 35 | 19.80 | Surfactant |
| Tween 80 | 28.08 | Surfactant |
| Soy/Rapeseed lecithin | 24.02 | Bio emulsifier |
| Propylene glycol | 13.10 | Co-surfactant/solvent |

According to at least one embodiment, the present invention further encompasses the microemulsion base to the saponified pigment ratio.

According to another embodiment, the present invention is a self-emulsifying microemulsion base that is capable of adopting a combination of two different microemulsion systems to formulate a highly concentrated microemulsion base.

By way of non-limiting example, in one embodiment, ethyl oleate/EL 35 and ethyl oleate/EL35-Tween 80 are suitable delivery system for carotenoids. Further still, the addition of short chain alcohol improves the formation of nano/microemulsion due to its ability to alter surfactant solubility characteristics. In order to reduce the high content of synthetic surfactant in the system, lecithin/lysolecithin can be optionally included as a bio-surfactant.

In at least one embodiment, the non-ionic surfactant is selected from the group consisting of polyoxyethylene, polyglycerol polyracinoleate, polysorbate (Tween 20, 40, 60, 65, 80, 85), and sorbitan monolaurate (SPAN 20 to 85). In at least one embodiment, the emulsifier is lecithin and/or lysolecithin (e.g., soya, egg, sunflower, rapeseed, rice bran) or polyoxyl castor oil (Cremophor EL)-10 to 80.

In one embodiment of the invention, the lecithin/lysolecithin based microemulsion includes at least one essential oil selected from the group consisting of methyl oleate, ethyl laurate, oleic acid, ethyl oleate, neem oil, thyme oil, clove oil, eugenol, cinnamon oil, eucalyptus oil, lemongrass oil, rose oil, lavender oil, carvacrol oil, and mixtures thereof.

According to at least one embodiment the inclusion of a lecithin/lysolecithin based microemulsion is desirable due to its natural origin and proven record of enhancing the absorption. Various studies have shown that the addition of lysolecithins in feed along with pigment products is able to improve pigment absorption in layers.

Another embodiment of the present invention relates to a dual-action microemulsion system, which is a novel, highly concentrated, self-emulsifying, bio-surfactant microemulsion base. According to at least one embodiment, the microemulsion base does not require an aqueous phase. This novel base formulation can be applied to saponified pigment, such as natural pigments, to form stable microemulsion utilizing the water present in the saponified pigment.

While persons of ordinary skill in the art may readily appreciate the myriad beneficial attributes of the present invention, it is worth briefly discussing certain aspects.

For instance, according to at least one embodiment, the present invention is a clear liquid comprising a combination of ethyl oleate, Tween 80, EL35, Soy/rapeseed lecithin (hydrolyzed and unhydrolyzed) and propylene glycol to create a microemulsion base. In certain embodiments, the microemulsion base is applied directly to saponified pigment to give an oil in water microemulsion.

In at least one embodiment, the present invention is a self-emulsifying microemulsion base that can be added at small amounts, for instance about 5-6%, to emulsify about 50% or more saponified pigment. This achievement offers the flexibility to use pigments without requiring water in the microemulsion base, instead utilizing the water content within the saponified pigment and in vivo to form the stable microemulsion.

In at least one embodiment, the present invention is a novel combination of one or more microemulsion systems. For instance, although EL35 may have better emulsifying capacity, persons of ordinary skill in the art would appreciate that it may have adverse effect in health due to synthetic surfactants. By replacing partial ethyl oleate/EL 35 and ethyl oleate/EL35-Tween 80 microemulsion with the hydrolyzed & unhydrolyzed lecithin microemulsion, the researchers were unexpectedly able to avoid the inclusion of high amount of synthetic surfactant similar to EL35.

In at least one embodiment, the present invention is a unique composition that contains only an oil phase to give a microemulsion base. Instead of typical microemulsions containing surfactants/oil phase and an aqueous phase, the present invention is a base formulated with surfactants/co-surfactant and ethyl oleate as the oil phase without requiring an aqueous phase. According to at least one embodiment, the aqueous phase is contributed by the saponified pigments. Without a pre-fixed volume for the aqueous phase, the present invention provides improved flexibility during application.

According to at least one embodiment, the composition comprises a microemulsion base containing ethyl oleate (long chain fatty acid ester as oil phase), EL35 and Tween 80, lecithin (hydrolyzed and unhydrolyzed), and propylene glycol (short chain alcohol co-surfactant) that emulsifies with the saponified pigment and improves its water solubility.

In alternative embodiments, the composition of the present invention may optionally include a bio emulsifier. In alternative embodiments, the composition may optionally include at least one essential oil. The components as described herein can be combined in any combination or in any order.

In at least one embodiment, the composition includes ethyl oleate in an amount ranging from about 10 to 50% by weight, for instance from about 13 to 40% or 12.5 to 30%, at least one ethoxylated non-ionic surfactant, such as EL 35 in an amount ranging from about 11.5 to 20.5%, for instance from about 12 to 20%, and Tween 80 in an amount ranging from about 16.5 to 29%, for instance from about 17 to 29%, a bio emulsifier such as soy/rapeseed lecithin in an amount ranging from about 14 to 25%, for instance from about 18 to 25%, and at least one co-surfactant, such as a short chain alcohol including but not limited to propylene glycol in an amount ranging from about 7.5 to 14%, for instance from about 10 to 14%.

According to at least one embodiment, the composition comprises a suitable microemulsion base with the ability to self-emulsify and form a stable microemulsion with moisture present in the saponified pigment.

According to at least one embodiment, the composition does not require a fixed amount of aqueous phase. In at least one embodiment, the composition does not include an aqueous phase. For instance, in at least one embodiment, the microemulsion base does not require the addition of water. In alternative embodiments, the microemulsion base may optionally include an aqueous phase or the presence of water.

According to at least one embodiment, the microemulsion base of the present invention is stable in the range of pH 2 to 12, such as for instance pH 4 to 10. In alternative embodiments, the microemulsion base is stable in the range of pH 5 to 9.

According to at least one embodiment, the microemulsion base of the present invention can be applied to emulsify products containing carotenoids and/or other water-insoluble nutrients. For instance, according to at least one embodiment, the microemulsion base can be used to emulsify products containing lutein, zeaxanthin, canthaxanthin, astaxanthin, cryptoxanthin, trans-capsanthin, capsorubin, violaxanthin, apo-carotenoids, and mixtures thereof.

In at least one embodiment, the compositions of the present invention are capable of being added or included in an amount ranging from about 5 to 8%, or in preferred embodiments about 5 to 6%, to emulsify at least 50% of the saponified pigment.

According to at least one embodiment, the saponified pigment contains total carotenoids in an amount ranging from about 60 to 90 g/kg, for instance 70 to 85 g/kg or 72 g/kg to 87 g/kg, and total lutein ranging from about 40 to 85 g/kg, for instance 45 to 80 g/kg or 50 g/kg to 74 g/kg.

According to at least one embodiment, the present invention relates to a process for preparing a microemulsion with the optional inclusion of long chain oil ethyl oleate in order to enhance the solubilizing capacity and lymphatic transport. It has been observed in various studies that drug lymphatic transport is oil chain-length dependent, and accordingly, long-chain oil provides greater enhancement.

At least one embodiment of the present invention relates to methods of improved dispersion of pigments in water. For instance, the self-emulsifying microemulsion base prototype of the present invention showed a greater degree of emulsification in water.

Because solubility in water is essential in greater absorption, the inclusion of the microemulsion base will improve the solubility and absorption of pigments in broilers and layers. For instance, one aspect of the present invention relates to incorporating the compositions of the present invention into animal feed to increase the color score of egg yolks. In alternative embodiments, the microemulsion base is used in aqua feed to increase the color intensity of the fish skin and scales. The invention could also potentially be used together with other water insoluble molecules.

Additional benefits of the present invention include, but are not limited to:
Highly emulsifying, i.e., able to self-emulsify more than 50% saponified pigment with at least 5% clear microemulsion base in final dry product. According to a preferred embodiment, the total composition of microemulsion is about 85 to 91% saponified pigment, such as 87, 88, 89, or 90% saponified pigment, about 5 to 10% water, such as 5, 6, 7, 8, 9, or 10% water, and about 8 to 14% microemulsion base, such as 8, 9, 10, 11, 12, 13, or 14% microemulsion base.
Able to disperse low water-soluble pigments in water and to form small micelles in oil and water with the addition of the present invention.
Preferential components, with partial replacement of ethyl oleate/EL 35 and ethyl oleate/EL35-tween 80 microemulsion with hydrolyzed & unhydrolyzed lecithin microemulsion to avoid the inclusion of high amount of synthetic surfactant whereas existing microemulsion contains no such ingredients.
Able to enhance lymphatic transport and solubilizing capacity.

### EXAMPLES

### Example 1: Dispersion Study Showed Improved Dispersion of Pigments in Water

The researchers evaluated a saponified pigment and the self-emulsifying microemulsion base (9:1 pigment to base). 5.95 g of the microemulsion base was added to 55.5 g of saponified pigment. The mixture was mixed thoroughly to ensure the formation of microemulsion, and 0.16 g of this sample was weighed and added into water. The mixture was left to stand for one hour. The control included a pigment without the inclusion of the microemulsion. Compared against the control, the researchers observed that the dispersion of pigment occurred without disturbance or agitation (FIG. 1).

The dispersion study showed that the novel microemulsion product was superior compared to the control for dispersion of pigment in water. The results indicate that the composition of the present invention is a suitable delivery system for carotenoids, including but not limited to lutein, zeaxanthin, canthaxanthin, astaxanthin, cryptoxanthin, trans-capsanthin, capsorubin, violaxanthin, apo-carotenoids, and mixtures thereof.

### Example 2: Particle Size Analysis

The researchers analyzed the particle size of the samples using a nano particle analyser, Horiba SZ-100Z, using methods readily known by those skilled in the art. Table 1 includes a summary of the analysis. Nanoemulsions are generally characterized by having a particle size in the range of approximately 10-100 nm. Based on results obtained from the analysis, the researchers were able to conclude that the formulated microemulsion prototype was indeed a nanoemulsion.

TABLE 2
Particle size analysis result

| Sample | Z-Average (nm) |
|---|---|
| Sap Conc without ME | 412.6 |
| | 285.0 |
| | 242.7 |
| Microemulsion (ME) | 51.5 |
| | 40.7 |
| | 42.4 |
| Sap. Conc with ME | 260.3 |
| | 271.4 |
| | 293.3 |

### Example 3: Observation of Small Micelles

To study the interactions of the present invention, the researchers analyzed the self-emulsifying microemulsion base with water and oil. Method: 1 g of the sample was prepared and added to 10 g of water and oil mixture (8:2 water: oil). The mixture then underwent 30 seconds of vortex and was immediately added to a slide. Results: The slides were observed under the optical microscope with 4× magnification (FIG. 2). The researchers observed that small micelles were visible in the sample that included the microemulsion base.

With reference to FIG. 2, it was observed that the new microemulsion base is able to emulsify and form small micelles (FIG. 2 c-d). Without the microemulsion base, the oil and phase exist as two separate phases. The researchers were able to conclude that the addition of the microemulsion base forms oil-in-water microemulsion.

### Example 4: Ternary Phase Diagram

A phase diagram was used to characterize the microemulsion and to determine the variables on the types of emulsion, oil-in-water (o/w) or water-in-oil (w/o), formed in the regions of the diagram. The phase diagram was constructed using ethyl oleate as the oil phase, water as the aqueous phase and EL/Tween 80/alcohol as the surfactant and co-surfactant. The various ratios (by weight) of the oil phase, water and surfactant/co-surfactant were plotted at the three phases of the diagram. The proportion of EL 35/Tween 80/short chain alcohol was fixed at weight ratio 1.98:2.81:2.4:1.31. Then 10 g of the three variables was added into a 15 ml conical tube and was vortexed for 60 min. After mixing, the mixture was placed at a stand for observation.

A phase diagram was constructed to study the relationship between water, oil phase, surfactants (EL35, Tween 80, phospholipids and propylene glycol) (FIG. 3).

### Example 5: Transmission Electron Microscopy (TEM)

Transmission electron microscopy (TEM) is a microscopy technique in which a beam of electrons is transmitted through a specimen to form an image. Prior to TEM analysis, the samples were diluted 50 times with deionized water. 20 μL of sample dropped onto a carbon coated copper grid for 30 sec. Excess sample was removed with a filter paper. Negative staining for 30 s with 20 μL of 2% uranyl acetate. Excess stain was removed with a filter paper before drying in a desiccator for overnight. The slide was observed under FEI Tecnai G2 Spirit Biotwin.

Uranyl acetate stain was used to enhance the viewing contrast by interacting the lipids with proteins. A total of 3 samples were prepared and the TEM imaging was recorded (FIG. 4-7). The researchers studied the TEM imaging of sap conc, sap conc with the new microemulsion base, and sap conc with the existing/previous microemulsion formula.

### Example 6: Particle Size Analysis

Samples were diluted with water and the particle size distribution was measured by the Malvern DLS particle analyzer. The particle size distribution results supported the observation made via TEM imaging. The sap conc has a smaller particle size when new ME has been added and a shift in the distribution curve was observed (FIG. 6).

### Example 7: Pigment Product with Microemulsion Quantum GLO 40Y Formulation

Marigold oleoresin with at least 150 g/kg total xanthophyll consisting of fatty acid esters of trans-lutein (80%) and zeaxanthin (5%), was set aside to produce Quantum GLO 40 as the finished good. Marigold oleoresin, an ester of carotenoid, was saponified according to the composition in Table 3 where free carotenoid and fatty acid derivatives were released. The microemulsion based was added to the saponified concentrate (sap conc) (Table 4) to allow the formation of microemulsion using the moisture present within the saponified pigment.

TABLE 3
The composition of saponified concentrate

| Ingredient | Composition (%) |
|---|---|
| Marigold oleoresin | 48.63 |
| Potassium hydroxide 45% | 20.94 |
| Liq | |
| Propylene glycol | 25.58 |
| Ethoxyquin 95% Liq | 4.45 |
| EDTA solution | 0.40 |

TABLE 4
The composition of Quantum GLO 40

| Ingredient | Composition (%) |
|---|---|
| Microemulsion | 5.95 |
| Sap conc | 55.53 |
| Silica | 37.00 |
| Ethoxyquin 95% Liq | 1.52 |

After which, the microemulsion-based saponified concentrate (sap conc) mixture was sprayed onto the dry silica carrier and powder product with Quantum GLO 40Y Dry with 40 g/kg Xanthophyl was produced, with a golden-orange color, as depicted in FIG. 7.

### Example 8: Animal (Layer) Trial by Quantum GLO 40Y (Pigment with Microemulsion) Supplemented Diet

Two hundred and eighty (280) layers of a commercial strain (Hy-Line brown) were allocated to 5 treatments, each treatment with 5 replications containing 8 birds as an experimental unit. A practical diet was formulated and used as the control diet. The test pigment products were supplemented in the control diet as summarized in Table 5.

TABLE 5
Treatment Design

| | | Sample | Feed | |
|---|---|---|---|---|
| Treatment | Diet | Code | Inclusion | |
| 1 | Control feed without | — | — | |
| | pigment supplementation | | | |
| 2 | Control feed with Oro GLO | Y1 | 1 | kg/t |
| | 20 Dry | | | |
| 3 | Control feed with Quantum | Y2 | 1 | kg/t |
| | GLO Y Dry | | | |
| 4 | Control feed with Quantum | Y3 | 0.5 | kg/t |
| | GLO 40Y Dry | | | |
| 5 | Control feed with Quantum | Y3 | 0.4 | kg/t |
| | GLO 40Y Dry | | | |

After feeding the pigment supplement for 6 weeks to birds, yolk color fan score, lutein content, egg weight, feed intake and feed conversion ratio (FCR) of different treatments were compared and the data is shown in Table 6. Lutein content analysis were done using High Performance Liquid Chromatography (HPLC).

The Yolk color fan (YCF) and lutein content after feeding pigment and non-pigment diet supplement after 6 weeks results are in Table 6.

TABLE 6
Yolk Color Fan (YCF) Score and Lutein
after supplementing (6 weeks)

| | | | | Lutein | Egg | Daily Feed |
|---|---|---|---|---|---|---|
| | Dosage | | YFC | content | weight | intake |
| Product | of feed | | score¹ | (mg/kg) | (g) | (g/bird/day) |
| Control | | | 1.00^{c} | 0.49^{c} | 61.29 | 110 |
| Oro GLO 20 Dry | 1 | kg/T | 5.95^{ab} | 3.61^{b} | 62.35 | 115 |
| Quantum GLO | 1 | kg/T | 6.33^{a} | 3.94^{b} | 62.98 | 110 |
| Y Dry | | | | | | |
| Quantum GLO | 0.5 | kg/T | 6.45^{a} | 4.62^{a} | 62.73 | 112 |
| 40Y Dry | | | | | | |
| Quantum GLO | 0.4 | kg/T | 5.44^{b} | 3.75^{b} | 60.17 | 113 |
| 40Y Dry | | | | | | |

^{a,b,c#}Means within column with no common superscript differ significantly [symbol] < 0 05;
¹ [symbol] colour was measured by Roche Yolk Colour Fan ™ [symbol] averaged score from three persons [symbol]
indicates data missing or illegible when filed

The results as summarized in Table 6 demonstrated that feeding of a product containing a pigment in birds has a clear effect on pigmentation with no adverse effect on the birds' health performance such as FCR, egg production, egg weight, mortality and feed intake. The Quantum GLO 40Y Dry at 0.5 kg/t has highest YCF score numerically but statistically similar to Quantum GLO Y Dry at 1 kg/t. The YCF scores of Quantum GLO 40Y Dry at 0.4 kg/t showed statistically difference from Quantum GLO Y Dry but no statically differences than Oro GLO 20 Dry at 1 kg/t feeding of the pigment supplement to the birds. The result showed that 0.4 kg/t (40%) Quantum GLO 40Y Dry (inclusion of new microemulsion) showed equal efficacy in YCF score as compared to 1 kg/t Oro GLO 20 Dry (non-microemulsion). The lutein content was plotted into a graph to compare the efficacy of the treatment as depicted in FIG. 8.

The bioavailability of lutein was evaluated for treatments Oro GLO 20 Dry and Quantum GLO Y Dry at 1 kg/t, Quantum GLO 40Y Dry 0.5 kg/t and Quantum GLO 40 Y Dry at 0.4 kg/t. The data is summarized in Table 7.

TABLE 7
Bioavailability of lutein content in egg yolk

| | | Feed | Lutein Inclusion | Lutein | Lutein in | Egg Yolk | |
|---|---|---|---|---|---|---|---|
| | Dose | Intake | (g in lutein/ | Intake | Egg Yolk | Weight | Bioavailability |
| Treatment | (Kg/T) | (g/Hen) | g in feed) | (g) | (mg/kg) | (g) | (%) |
| Control | — | 110 | — | — | 0.49 | 22.46 | — |
| Oro GLO 20Y | 1 | 115 | 0.000016 | 0.00184 | 3.61 | 22.713 | 3.85 |
| Quantum GLO Y | 1 | 110 | 0.000016 | 0.00176 | 3.94 | 22.797 | 4.47 |
| Quantum GLO 40Y | 0.5 | 112 | 0.000016 | 0.001792 | 4.62 | 22.844 | 5.26 |
| Quantum GLO 40Y | 0.4 | 113 | 0.0000128 | 0.001446 | 3.75 | 22.197 | 5.00 |

The results as summarized in Table 7 showed that Quantum GLO 40Y Dry has higher bioavailability than Quantum GLO Y Dry and Oro GLO 20 Dry. This demonstrated that Quantum GLO 40Y Dry at 0.4 kg/t showed higher lutein bioavailability than Quantum GLO Y Dry and significantly higher than Oro GLO 20 Dry.

Finally, some of the collected eggs were used for color assessment from all the treatments as depicted in FIG. 9. For instance, clear differences were observed on yolk color between Oro GLO 20 Dry and other treatments at 1 kg/t of Quantum GLO Y Dry, Quantum GLO Y Dry, Quantum GLO 40 Y Dry at 0.5 kg/t and Quantum GLO 40Y Dry at 0.4 kg/t.

### Example 9: Commercial Farm Feedback on Broiler Skin Color after Using Quantum GLO 40 Y Dry

The efficacy of Quantum GLO 40 was studied by customer by replacing competitor product and replacing Oro GLO 20 Dry, a non-microemulsion product. Based on the feedback from customers, the Quantum 40Y Dry showed more enhanced broiler skin and shanks color as compared to competitor product as in Table 8:

TABLE 8
Customer feedback of broiler skin color efficacy after
using Quantum GLO 40 Y Dry in the commercial farm.

| | | Feedback from |
|---|---|---|
| Chicken | How Quantum GLO 40Y | customer |
| Species | was used? | evaluation |
| Native | 0.5 kg/MT of Quantum GLO | Will continue to |
| Chicken | 40Y Dry replaced 1 kg/MT | use as the QG40 Y |
| | of Oro Glo 20 Dry in | Dry showed good |
| | Native Chicken | performance. |
| Native | Commercial trial: 1:1 | Will continue to |
| Chicken | replacement of Xamacol | use since the |
| | 40 [40 g/kg] by Quantum | QG40 Y Dry showed |
| | GLO 40Y | good performance. |
| | Pigment Supplement Period: | The broiler skin |
| | 4^{th}~9^{th} weeks | and shanks color |
| | Pigment addition amount: | appeared more |
| | Quantum GLO 40Y: 2 kg/MT | enhanced when |
| | and Carophyll Red: | compared using |
| | 50 g/MT of feed. | Xamacol 40. |
| | Pigment Supplement Period: | Quantum GLO 40Y |
| | 10^{th}~12^{th} week | Dry showed better |
| | Pigment addition amount: | product stability. |
| | Quantum GLO 40Y at 1 kg/MT | |
| | and Carophyll Red: | |
| | 100 g/MT of feed. | |
| | Skin color measured on week 13^{th} | |
| | before selling the chicken. | |
| | COLOR FAN SCORE on 13th week: | |
| | Between 10 to 11. | |

### Example 10: Layers Commercial Farm Feedback after Using Quantum GLO 40 Y Dry

The mixture of Kem GLO Dry and Quantum GLO 40 in 2:1 was supplemented to 30,000 bird of species HENDRIX Brown Layer at Changhua County Egg Farm, Taiwan for a period between 31 to 37 weeks. Based on customer's feedback, the lutein content increased by 200 g in egg yolk after feeding with Quantum GLO 40 Y Dry for a week. Customer noted a higher lutein accumulation compared to the previous yellow pigment product (Oro Glo 20 Dry) due to better pigment absorption.

### Example 11: Heat Stability Studies for Pelleting Condition

Stability of Quantum GLO 40 was studied at high temperature to ensure that the product stability is maintained during the feed pelleting process. To study the pelleting temperature stability, 6 temperature points were selected: 25° C., 75° C., 85° C., 95° C., 105° C., 115° C. Samples were placed in the oven for 10 minutes after the oven has reached the set temperature. Subsequently, recovery of xanthophyll in the samples was then measured; recovery was conducted in triplicates. The results showed that Quantum GLO 40 with the new microemulsion was stable up to 115° C. for 10 mins with a recovery of 99.03% as shown in FIG. 10.

Having described the invention with reference to specific compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A microemulsion base comprising a long chain fatty acid, at least one non-ionic surfactant, a bio emulsifier, and at least one co-surfactant, wherein the microemulsion base does not require the addition of water.

2. The microemulsion base of claim 1, wherein the long chain fatty acid is ethyl oleate.

3. The microemulsion base of claim 1, wherein the at least one non-ionic surfactant is selected from the group consisting of polyoxyethylene, polyglycerol polyracinoleate, polysorbate (Tween 20, 40, 60, 65, 80, 85), and sorbitan monolaurate (SPAN 20 to 85).

4. The microemulsion base of claim 1, wherein the bio emulsifier is lecithin, lysolecithin, or a mixture.

5. The microemulsion base of claim 1, wherein the at least one co-surfactant is a short chain alcohol.

6. The microemulsion base of claim 1 wherein the at least one co-surfactant is selected from the group consisting of glycerol, ethanol, propanol, isopropanol, butanol, n-pentanol, hexanol, sorbitol, n-pentanoic acid, n-hexanoic acid, n-butylamine, sec-butylamine, 2-aminopentane, 1,2-butanediol, propylene glycol, and glycerol.

7. The microemulsion base of claim 1, comprising ethyl oleate as the oil phase in an amount ranging from about 12.5 to 50%, a first non-ionic surfactant in an amount ranging from 11.5 to 20.5%, a second non-ionic surfactant in an amount ranging from 16.5 to 29%, lecithin in an amount ranging from 14 to 25%, and propylene glycol in an amount ranging from 7.5 to 14%, by weight.

8. The microemulsion base of claim 1 further comprising at least one essential oil selected from the group consisting of methyl oleate, ethyl laurate, oleic acid, ethyl oleate, neem oil, thyme oil, clove oil, eugenol, cinnamon oil, eucalyptus oil, lemongrass oil, rose oil, lavender oil, carvacrol oil, and mixtures thereof.

9. The microemulsion base of claim 1, further comprising at least one acid is selected from the group consisting of propionic acid, formic acid, and lactic acid.

10. The microemulsion base of claim 1, wherein the microemulsion base is stable in the range of pH 2 to 12.

11. The microemulsion base of claim 1, which is further combined with at least one carotenoid, pigment and/or other water-insoluble bioactive ingredient or nutrient.

12. A method of delivering at least one carotenoid, pigment and/or other water-insoluble bioactive ingredient or nutrient to an animal comprising combining:
a microemulsion base that contains an oil phase, one or more non-ionic surfactants, one or more co-surfactants, and a bio emulsifier,
with the at least one carotenoid, pigment and/or other water-insoluble bioactive ingredient or nutrient.

13. The method of claim 12, wherein the oil phase is ethyl oleate.

14. The method of claim 12, wherein the microemulsion base is added in an amount ranging from about 5 to 8% by weight in order to emulsify at least 50% of the at least one carotenoid, pigment and/or other water-insoluble bioactive ingredient or nutrient.

15. The method of claim 16, wherein the carotenoid is selected from the group consisting of lutein, zeaxanthin, canthaxanthin, astaxanthin, cryptoxanthin, trans-capsanthin, capsorubin, violaxanthin, apo-carotenoids, and mixtures thereof.

16. The method of claim 17, wherein the saponified pigment contains total carotenoids from about 72 to 87 g/kg and total lutein ranging from about 50 to 74 g/kg.

17. A method of increasing absorption of pigments or carotenoids by a fish or animal comprising administering to the fish or animal a microemulsion that includes the microemulsion base of claim 1 and a saponified pigment.

18. The method of claim 19, wherein the composition is incorporated into a poultry diet and results in an increased egg yolk color score.

19. The method of claim 19, wherein the composition is incorporated into an aqua diet and results in an increased intensity of the exterior pigment of the fish.

20. The method of claim 6 wherein the saponified pigment contains total carotenoids from 72 g/kg to 87 g/kg and total lutein ranging from 50 g/kg to 74 g/kg.
